(19) United States
(12) Patent Application Publication
(10) Pub. No.: US 2019/0285642 A1
(43) Pub. Date: Sep. 19, 2019

(54) COMPUTER-IMPLEMENTED METHODS OF ESTIMATING A PROBABILITY OF ECTOPIC PREGNANCY IN A SUBJECT, COMPUTER-READABLE MEDIA, AND METHODS OF DIAGNOSING AND TREATING A SUBJECT PRESENTING WITH A PREGNANCY OF UNKNOWN LOCATION (PUL)

(71) Applicant: Women & Infants Hospital of Rhode Island, Providence, RI (US)

(72) Inventors: Ruben Alvero, Los Altos, CA (US); Shunping Wang, Barrington, RI (US); Yen-Tsung Huang, Taipei (TW); Yi (Jamie) Zhang, Sunnyvale, CA (US); Reeva B. Makhijani, Wallington, NJ (US)

(21) Appl. No.: 16/296,933

(22) Filed: Mar. 8, 2019

Related U.S. Application Data
(60) Provisional application No. 62/642,820, filed on Mar. 14, 2018.

(51) Int. Cl. G01N 33/68; G01N 33/53; G06N 7/00; G06F 17/18

(52) U.S. Cl. CPC G01N 33/689; G06F 17/18; G06N 7/005; G01N 33/53

(57) ABSTRACT

One aspect of the invention provides a method of diagnosing and treating a subject presenting with a pregnancy of unknown location (PUL). The method includes: obtaining a parameter set including: a first β-human chorionic gonadotropin (β-hCG) value generated using a first sample from a subject; and a second β-human chorionic gonadotropin (β-hCG) value generated using a second sample from a subject, wherein the second sample is obtained between about 36 hours and about 72 hours after the first sample; providing the parameter set as an input to either a computer-implemented method as described herein or a computer executing the program instructions of a non-transitory computer readable medium as described herein; receiving a probability of ectopic pregnancy in the subject; and treating the subject based on the probability.

### CROSS-REFERENCE TO RELATED APPLICATION

This application claims the benefit of priority to U.S. Provisional Patent Application Ser. No. 62/642,820, filed Mar. 14, 2018. The entire content of this application is hereby incorporated by reference herein.

### BACKGROUND OF THE INVENTION

The incidence of ectopic pregnancy (EP), a gestation that is implanted outside of the endometrial cavity, is 1-2% in the general population. As many as 25-50% of EPs present as pregnancies of unknown location (PUL) and 6-20% of women who present to emergency rooms with PULs are ultimately diagnosed with EPs. Fortunately, morbidity and mortality associated with EP has significantly decreased due to earlier detection and treatment before rupture. This, in turn, has led to a paradigm shift, in which EPs are no longer seen as a fatal condition, but rather a benign issue often found in asymptomatic women. Nonetheless, ruptured EPs still occur and are the cause of 3-4% of pregnancy-related maternal deaths due to intraabdominal hemorrhage leading to shock and ultimately to cardiopulmonary arrest.

### SUMMARY OF THE INVENTION

One aspect of the invention provides a computer-implemented method of estimating a probability of ectopic pregnancy in a subject. The computer-implemented method includes: receiving a parameter set including: a first β-human chorionic gonadotropin (β-hCG) value generated using a first sample from a subject; and a second β-human chorionic gonadotropin (β-hCG) value generated using a second sample from a subject, wherein the second sample is obtained between about 36 hours and about 72 hours after the first sample; calculating a ratio the second (β-hCG value to the first β-hCG value to produce an augmented parameter set including: the first β-hCG value; and the ratio of the second (β-hCG value to the first β-hCG value; and solving a generalized additive model for a probability of ectopic pregnancy in the subject based the augmented parameter set, wherein the generalized additive model was previously backfit with a data set of subject data including the augmented parameter set for each subject in the data set, thereby estimating the probability of ectopic pregnancy in the subject.

This aspect can have a variety of embodiments. The generalized additive model can be a penalized spline generalized additive model. The generalized additive model can be a penalized log-likelihood generalized additive model.

The parameter set and the augmented parameter set can further include one or more risk factors. The one or more risk factors can include one or more selected from the group consisting of: history of prior ectopic pregnancy, history of pelvic inflammatory disease, history of tubal ligation, presence of intrauterine device, history of diethylstilbestrol (DES) exposure, history of infertility, history of pelvic surgery, and history of sexually transmitted infections.

The parameter set and the augmented parameter set can further include one or more demographics. The one or more demographics can include one or more selected from the group consisting of: age and parity.

The parameter set and the augmented parameter set can further include total time followed by medical professionals.

The parameter set and the augmented parameter set can further include total number of β-hCG values measures.

The first sample and the second sample can both be selected from the group consisting of: blood, blood serum, blood plasma, and urine.

The first β-hCG value to the second β-hCG value can be determined using a sandwich assay.

The first sample and the second sample can both be urine and the first β-hCG value and the second β-hCG value can be determined using an assay selected from the group consisting of: a chromatographic immunoassay and a lateral flow assay.

The first sample and the second sample can both be blood serum and the first β-hCG value and the second β-hCG value can be determined using an assay selected from the group consisting of: a chemiluminescent immunoassay or fluorimetric immunoassay.

This aspect of the invention can have a variety of embodiments. The subject can be a mammal. The subject can be a human female.

Another aspect of the invention provides a non-transitory computer readable medium containing program instructions executable by a processor. The computer readable medium includes program instructions to implement a computer-implemented methods as described herein.

Another aspect of the invention provides a method of diagnosing and treating a subject presenting with a pregnancy of unknown location (PUL). The method includes: obtaining a parameter set including: a first β-human chorionic gonadotropin (β-hCG) value generated using a first sample from a subject; and a second β-human chorionic gonadotropin (β-hCG) value generated using a second sample from a subject, wherein the second sample is obtained between about 36 hours and about 72 hours after the first sample; providing the parameter set as an input to either a computer-implemented method as described herein or a computer executing the program instructions of a non-transitory computer readable medium as described herein; receiving a probability of ectopic pregnancy in the subject; and treating the subject based on the probability.

This aspect of the invention can have a variety of embodiments. The subject can be a mammal. The subject can be a human female.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 is a flow diagram depicting a process of screening data for analysis according to an embodiment of the invention.

FIGS. 2A and 2B depict generalized additive models (GAMs) according to embodiments of the invention. FIG. 2A depicts a 2-dimensional view with β-hCG ratio on x-axis and probability of EP on y-axis. FIG. 2B depicts a 3-dimensional view with β-hCG ratio on x-axis, baseline β-hCG on y-axis and probability of ectopic pregnancy (EP) on z-axis. No risk factors are included in FIG. 2B.

FIG. 3 depicts ROC curves for: X (baseline (β-hCG alone (AUC=0.793)); Y (β-hCG ratio alone (AUC=0.88)); and Z (baseline (β-hCG and (β-hCG ratio in combination (AUC=0.889)).

FIG. 4 depicts a computer-implemented method 400 of estimating a probability of ectopic pregnancy in a subject according to an embodiment of the invention.

FIG. 5 depicts a method 500 of diagnosing and treating a subject presenting with a pregnancy of unknown location (PUL) according to an embodiment of the invention.

FIG. 6 depict a user-interface for an implementation of a computer-implemented method of estimating a probability of ectopic pregnancy in a subject according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

The terms “subject” and “patient” are used interchangeably and include any live-bearing member of the class mammalia, including humans, domestic and farm animals, and zoo, sports or pet animals, such as mouse, rabbit, pig, sheep, goat, cattle, horses and higher primates. In certain embodiments, the subject/patient is a human female.

### DETAILED DESCRIPTION OF THE INVENTION

PULs pose a number of diagnostic challenges. First, ectopic pregnancies often present with vague symptoms, which providers may not initially recognize. This can result in a ruptured ectopic pregnancy. Furthermore, although risk factors of EP, such as the history of a prior EP or pelvic inflammatory disease, are commonly known, many EPs occur in patients without any risk factors. Typically, patients with PULs are closely monitored until a diagnosis of intrauterine pregnancy (IUP), spontaneous abortion or miscarriage (SAB) or EP is reached. Yet, despite extensive research on the diagnosis and management of EPs and, specifically, on the predicted rates of change in β-hCG, multiple studies have shown that lab monitoring alone does not replace clinical judgment, as EP can occur even in cases of appropriately rising or falling levels of (β-hCG.

Given these challenges associated with identifying EPs, it remains difficult to objectively determine the degree of follow up that is necessary for each patient. At Applicant's institution, patients who present to the emergency room with a PUL are followed in a resident-run Beta Book procedure, which is a common mechanism used by OB/GYN residents nationally to follow PULs. It employs strict algorithms for following these patients until their pregnancies are located. This degree of follow-up can be quite onerous and reaching a diagnosis can take up to 6 weeks.

Better tools to more accurately and efficiently diagnose ectopic pregnancies are needed. Although morbidity and mortality have decreased, the emphasis should now be on creating tools that optimize follow up and allow for better allocation of time and resources. No existing decision tools have sufficient specificity and sensitivity to obviate the need for close follow up. This study aims to take the data collected in the Beta Book, which includes information about the patient's clinical history and presentation as well as trends in β-hCG levels, to create a comprehensive, simple, accurate, and non-invasive risk assessment tool for the prediction of ectopic pregnancies. Novel modeling techniques are also used to more accurately predict risk for ectopic pregnancy.

### Computer-Implemented Method of Estimating Probability of Live Birth in a Subject

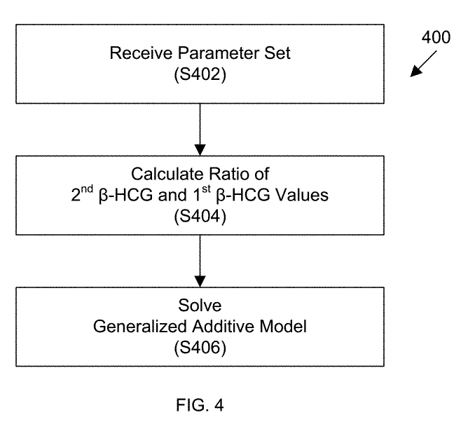

Referring now to FIG. 4, one aspect of the invention provides a computer-implemented method 400 of estimating a probability of ectopic pregnancy in a subject.

In step S402, a parameter set is received. The parameter set can include a first and a second β-human chorionic gonadotropin (β-hCG) value generated using time-separated samples from the subject. In one embodiment, the second sample is obtained between about 36 hours and about 72 hours after the first sample. Exemplary samples include: blood, blood serum, blood plasma, urine, and the like. The first and second β-hCG values can be obtained using techniques such as sandwich assays, chromatographic immunoassays, lateral flow assays, chemiluminescent immunoassays, fluorimetric immunoassays, and the like.

The β-hCG values can be generated locally (e.g., within the laboratory of a clinic or hospital) or can be obtained from an external clinical laboratory service such as, e.g., Quest Diagnostics Incorporated of Madison, N.J.

The parameter set and the augmented parameter set can include additional data beyond β-hCG values such as risk factors (e.g., history of prior ectopic pregnancy, history of pelvic inflammatory disease, history of tubal ligation, presence of intrauterine device, history of diethylstilbestrol (DES) exposure, history of infertility, history of pelvic surgery, and history of sexually transmitted infections), demographics (e.g., age, parity (the number of times a female has given birth), and the like), total time followed by medical professionals, total number of β-hCG values measures, and the like.

In step S404, a ratio of the second β-hCG value to the first β-hCG value is calculated to product an augmented parameter set including the first β-hCG value and the ratio.

In step S406, a generalized additive model is solved for a probability of ectopic pregnancy in the subject based on the augmented parameter set. The generalized additive model (GAM) was previously backfit with a data set of subject data including the augmented parameter set for each subject in the data set. For example, the GAM can be backfit using the R™ programming language and software environment for statistical computing and graphics provided by the R Foundation for Statistical Computing (Boston, Mass.). The GAM can be a penalized spline GAM and/or a penalized log-likelihood GAM.

Method of Diagnosing and Treating a Subject Presenting with a Pregnancy of Unknown Location (PUL)

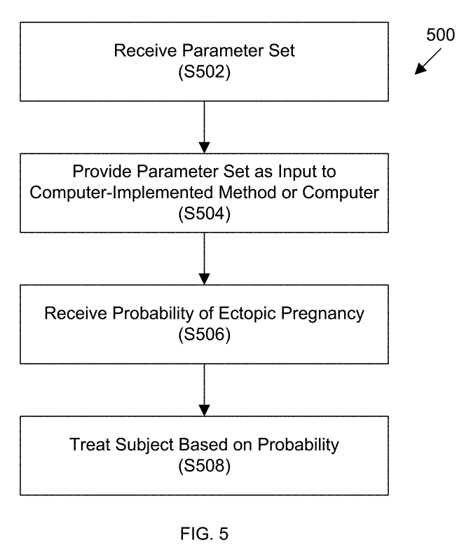

Referring now to FIG. 5, another aspect of the invention provides a method 500 of diagnosing and treating a subject presenting with a pregnancy of unknown location (PUL).

In step S502, a parameter set is received. The parameter set can include a first and a second β-human chorionic gonadotropin (β-hCG) value generated using time-separated samples from the subject, e.g., as described in the context of step S402.

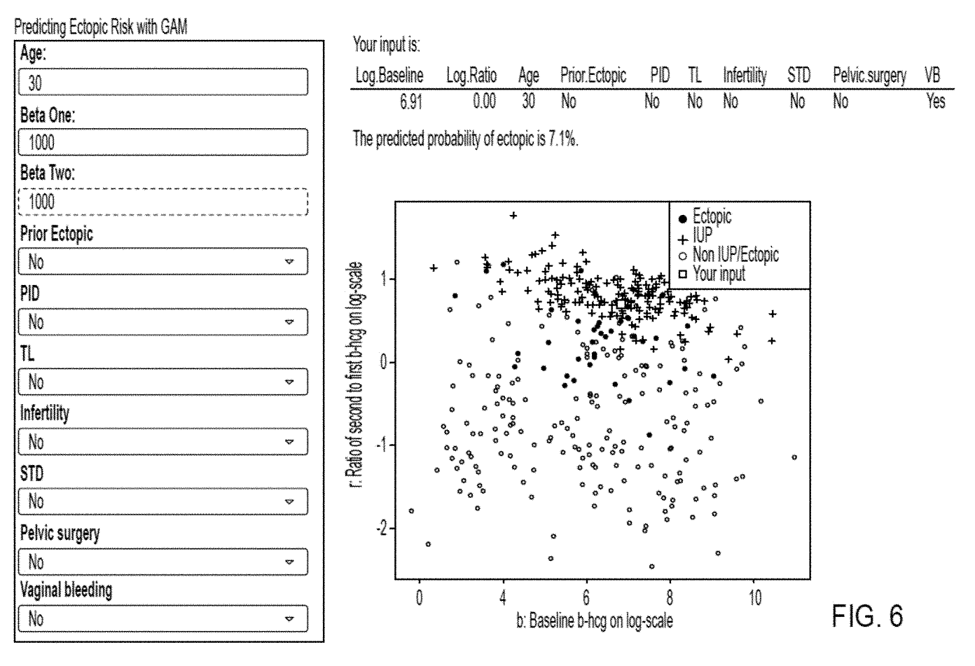

In step S504, the parameter set is provided as an input to a computer-implemented method as described herein (e.g., computer-implemented method 400) or a computer executing program instructions (e.g., stored in computer-readable media). For example, the parameter set can be input by a healthcare professional, e.g., using a computer (e.g., a desktop, a laptop, a tablet, a smartphone, and the like). In another embodiment, the parameter set can be provided electronically by a laboratory or the methods described herein can be performed by the laboratory after generating the parameter set. An exemplary graphic user interface (GUI) is depicted in FIG. 6.

In step S506, a probability of ectopic pregnancy in the subject is received. The probability can be expressed in a variety of forms such as percentages, ratios, fractions, and the like. The probability can be provided electronically (e.g., as an input to the infertility subject's electronic medical record, via e-mail, via a secure portal), via a printed report, aurally, and the like.

In step S508, the subject is treated based on the probability. For example, various probability ranges can indicate use of a particular treatment. In one embodiment, subjects having a probability of an ectopic pregnancy above a threshold (e.g., about 15%) can be monitored more closely (e.g., using the Beta Book protocol) than patients having a probability below the threshold. Exemplary treatments for a likely ectopic pregnancy include administration of methotrexate and surgical intervention.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### Working Example

### Materials and Methods

### Data Collection

All women with PULs who were followed in a resident-run Beta Book procedure at a single academic institution (Women & Infants Hospital, Providence, R.I.) between September 2008 and October 2016 were eligible for screening. Patients were followed in the Beta Book procedure if they were diagnosed with a PUL and were patients of the OB/GYN resident clinic service or did not have a primary gynecologist in the community at the time of presentation. A PUL was defined as having a positive urine or serum β-hCG, but no signs of intra- or extra-uterine pregnancy on transvaginal ultrasound. Retrospective review of 800 electronic medical records was performed. To be included, the patient had to have at least 2 peripheral serum quantitative β-hCG measurements that were collected between 36 to 72 hours apart. Patients were excluded if: (i) the time interval between the first and second β-hCG was not known; (ii) the time interval between the first and second β-hCG was <36 hours or >72 hours; (iii) the patient ultimately was diagnosed with a molar pregnancy; (iv) the patient had a ruptured ectopic pregnancy and had surgery before the second β-hCG was measured; and/or (v) the patient had no outcome (IUP v. SAB v. EP) recorded.

Data collected included demographics (age, parity), presenting symptoms (vaginal bleeding, abdominal pain), presence of risk factors for ectopic pregnancy (history of prior EP, history of pelvic inflammatory disease, history of tubal ligation, presence of intrauterine device, history of diethylstilbestrol (DES) exposure, history of infertility, history of pelvic surgery, history of sexually transmitted infections), total time followed, total number of β-hCG values measures, β-hCG values with time/date of collection and diagnosis (EP v. SAB v. IUP).

An intrauterine pregnancy was diagnosed once a yolk sac or fetal pole was visualized on ultrasound. A spontaneous abortion or miscarriage was diagnosed if there was appropriately down trending β-hCG or inappropriately rising or falling β-hCG and pathology specimen after uterine evacuation procedure was consistent with products of conception or the diagnosis of spontaneous abortion was presumed if β-hCG values declined >50% over 48 hours. Lastly, a diagnosis of EP was presumed if (i) β-hCG plateaued or continued to rise after uterine evacuation procedure; (ii) if β-hCG was greater than the discriminatory zone (at Applicant's institution, 3000 mIU/mL) and the uterus did not show an intrauterine pregnancy, but evidence of an extra-uterine mass adjacent to but separate from the ovary or an extra-uterine mass with a fetal pole with or without fetal cardiac activity.

All data were collected and managed using REDCAP® electronic data capture tools hosted at Women & Infants Hospital. After data was collected, it was validated by re-extracting 10% of randomly selected records.

### Model Building

A statistical model was built to characterize the association of the β-hCG measured at two time points with the outcome of ectopic pregnancy. Applicant transformed the two β-hCG measures into an initial β-hCG value and a ratio of the second β-hCG value to the first β-hCG value. The first and second β-hCG levels were measured 36-72 hours apart. Such a transformation preserved equivalent information to the original two measurements and provided better clinical relevance and utility. In order to capture the non-linear relationship of β-hCG levels and the risk of ectopic pregnancy, Applicant fitted a generalized additive model (GAM), in which semiparametric penalized splines were used to depict the two-dimensional profile of first β-hCG value and the ratio in relation to the outcome of ectopic pregnancy. This type of model was used because it does not assume any type of relationship between independent and dependent variables, thereby allowing for the model to reveal hidden relationships, while also avoiding overfitting. Throughout the statistical modeling, variables known to be risk factors for ectopic pregnancy or significantly associated with the risk of EP (p<0.05) were adjusted as covariates.

### Statistical Analysis and Performance Measures

Baseline characteristics were analyzed using Student t-test for continuous variables and Fischer's exact test for categorical variables (p<0.05).

Prediction performance was assessed with receiver operating characteristics (ROC) curves of various candidate GAM models, including initial β-hCG alone, β-hCG ratio alone, and the two combined. The area under the ROC curve (AUC) was calculated to analytically compare across models. Applicant selected the model with the largest AUC and, from the ROC curve, obtained cut-offs for predicting the occurrence of ectopic pregnancy by balancing the sensitivity and specificity. For a given cut-off, Applicant was also able to calculate positive predictive value (PPV) and negative predictive value (NPV), provided that the prevalence of ectopic pregnancy was known.

### Results

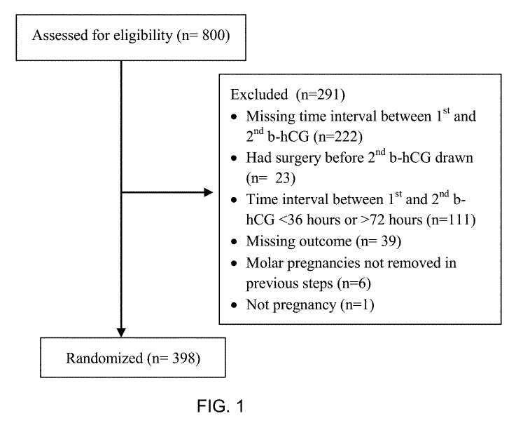

Electronic medical records for 800 patients followed in the Beta Book procedure were screened for eligibility. After all exclusions were made, data from 398 patients were ultimately included in the analysis (FIG. 1).

A total of 40 patients were diagnosed with ectopic pregnancy (10.1%), 190 with intrauterine pregnancy (47.7%), and 168 with spontaneous abortion (42.4%). Patients diagnosed with ectopic pregnancy were more likely to be followed for a longer period of time (41.5±25.0 days v. 11.6±9.3 days, p<0.05) and have more β-hCG levels measured (9.9±4.1 v. 3.0±1.5, p<0.05). Patients ultimately diagnosed with ectopic pregnancy also more often had a history of a prior ectopic pregnancy (17.5% v. 4.5%, p<0.05) or a history of pelvic surgery (12.5% v. 3.6%, p<0.05). They were also more likely to present with vaginal bleeding (85.0% v. 55.6%, p<0.05). Table 1 provides a description of the cohort.

TABLE 1
Demographic Characteristics and Baseline Features of the Cohort

| Table I: Cohort | All subjects | Ectopic | Not Ectopic | |
|---|---|---|---|---|
| Characteristics | (n = 398) | (n = 40) | (n = 358) | □ = 0.05 |
| Age (years) | 27.25 ± 6.24 | 27.78 ± 5.61 | 27.20 ± 6.31 | 0.58 |
| Average time period followed | 14.60 ± 14.84 | 41.55 ± 24.96 | 11.58 ± 9.29 | 0.0001 |
| (days) | | | | |
| Average time to diagnosis | 15.66 ± 43.41 | 6.93 ± 3.26 | 16.63 ± 45.67 | 0.18 |
| (days) | | | | |
| Parous (y/n) | 224/398 | 20/40 | 204/358 | 0.41 |
| Primary presenting symptom | | | | |
| Vaginal bleeding | 233/398 | 34/40 | 199/358 | 0.0003 |
| Abdominal pain | 284/398 | 25/40 | 259/358 | 0.20 |
| Risk Factors | | | | |
| h/o prior ectopic (y/n) | 23/398 | 7/40 | 16/358 | 0.0045 |
| h/o PID | 7/398 | 2/40 | 5/358 | 0.15 |
| h/o TL | 3/398 | 1/40 | 2/358 | 0.27 |
| h/o IUD | 4/498 | 0/40 | 4/358 | 1.0 |
| h/o DES exposure | 0/398 | 0/40 | 0/358 | 1.0 |
| h/o infertility | 9/398 | 2/40 | 7/358 | 0.23 |
| h/o STD | 34/398 | 7/40 | 27/358 | 0.0647 |
| h/o Pelvic Surgery | 20/398 | 5/40 | 15/358 | 0.0397 |
| Average total # TVUS | 1.57 ± 0.80 | 1.60 ± 0.72 | 1.56 ± 0.80 | 0.76 |
| performed | | | | |
| Average total β-hCG values | 3.71 ± 2.85 | 9.9 ± 4.11 | 3.01 ± 1.54 | 0.0001 |
| measured | | | | |

TABLE 2
Diagnoses

| | |
|---|---|
| Ectopic | 40/398 (10.1%) |
| SAB | 190/398 (47.7%) |
| IUP | 168/398 (42.2%) |

### GAM Model

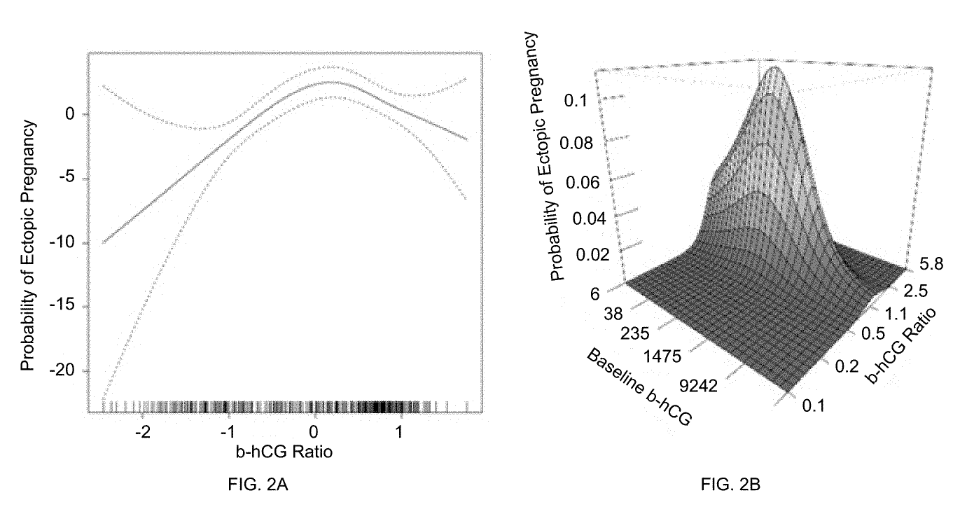

Generalized additive models (GAMs) were used to investigate the nonlinear fixed effects of β-hCG ratio and the probability of ectopic pregnancy using penalized spline as depicted in FIG. 2A. (Penalized spline modeling is further described in S. N. Wood, “Thin plate regression splines”, 65(1) J. Royal Statistical Soc.: Series B (Statistical Methodology) 95-114 (2003).) The data suggest that β-hCG ratio alone provides a reasonable prediction of ectopic pregnancy.

Applicant further investigated whether the joint effects of baseline β-hCG and β-hCG ratio were able to offer a more accurate prediction. This analysis was performed using two-dimensional spline under GAM. The model was fitted through maximizing a penalized log-likelihood using R™ package mgcv. Based on the fitted models, Applicant was able to predict the probability of ectopic pregnancy for a certain patient given her baseline β-hCG and β-hCG ratio. To visualize the dose-response relationship of baseline β-hCG and β-hCG ratio with respect to the probability of ectopic pregnancy, Applicant plotted predicted probabilities given the corresponding baseline β-hCG and β-hCG ratio, as shown in FIG. 2B. The yellow and orange areas in the figure suggest a higher probability of ectopic pregnancy, corresponding to a certain range of baseline β-hCG and β-hCG ratio.

### ROC Curve Analysis

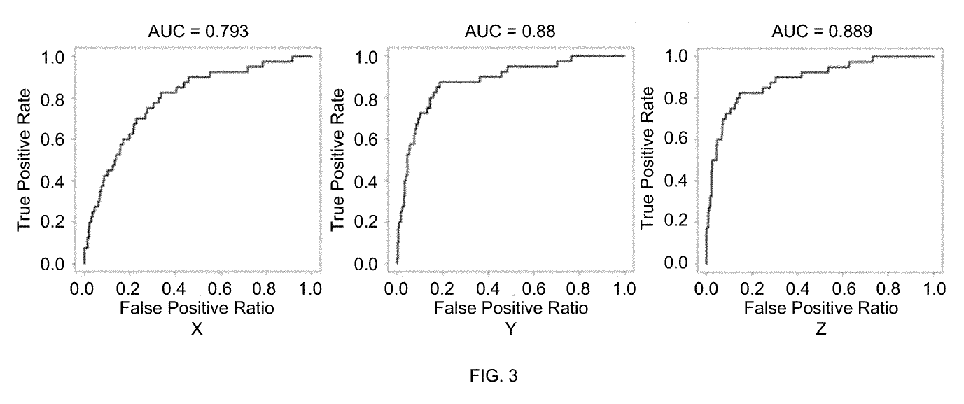

Given that EP was the primary outcome of interest, Applicant performed ROC analysis by combining the other two categories (SAB and IUP) into one group. Applicant created ROC curves for (X) baseline β-hCG alone, (Y) β-hCG ratio alone, and (Z) baseline β-hCG and β-hCG ratio in combination. FIG. 3 shows the ROC curves, each with their AUC reported. X gave an AUC of 0.793 for predicting ectopic pregnancy. Y gave an AUC of 0.88 and Z gave an AUC of 0.889.

From the best performing model, Z, Applicant computed the sensitivities, specificities, positive predictive values and negative predictive values for a range of risk cut-offs for ectopic pregnancy (5-40% in 5% increments). The highest sensitivity and specificity was seen at a cut-off score of 15%. These performance measures are displayed in Table 3.

TABLE 3
Performance Measures for ROC Curve ‘Z’

| Ectopic Pregnancy Risk Cut-Off | | | | |
|---|---|---|---|---|
| (%) | Sensitivity | Specificity | PPV | NPV |
| 5% | 0.9 | 0.656 | 0.226 | 0.983 |
| 10% | 0.825 | 0.765 | 0.282 | 0.975 |
| 15% | 0.825 | 0.852 | 0.384 | 0.978 |
| 20% | 0.75 | 0.877 | 0.405 | 0.969 |
| 30% | 0.6 | 0.953 | 0.585 | 0.955 |
| 40% | 0.425 | 0.978 | 0.68 | 0.938 |

### DISCUSSION

Once a diagnosis of ectopic pregnancy is made, the management is fairly straightforward. However, clinicians face a diagnostic dilemma when a patient presents with a positive pregnancy test and the pregnancy cannot be located on transvaginal ultrasound. The management of pregnancies of unknown location are challenging because there is a tension between allowing a normal pregnancy to continue versus preventing an adverse event. In both cases, the pregnancy needs to evolve until reasonable diagnostic accuracy is achieved.

Moreover, algorithms are variable and do not take into consideration each patient's complex clinical situation. For example, a woman who presents with vaginal bleeding or abdominal pain in the setting of positive pregnancy test has a higher pre-test probability for having an ectopic pregnancy than a patient who is asymptomatic. Similarly, a person who becomes pregnant after having a tubal ligation is at significantly higher risk than a patient without any risk factors.

Although the risk factors for EP are well-characterized and most clinicians are aware of what factors may raise or lower clinical suspicion, there are no tools to objectively quantify an individual patient's risk that take into account the full and heterogeneous nature of a patient's presentation and clinical history. Thus, Applicant's focus was on identifying a way to make a comprehensive and objective assessment of a patient's risk of EP in order to simplify follow-up and allocate resources and time to those patients at highest risk.

A GAM model, unlike multiple linear regression, offers the advantage of elucidating the relationships between each of the independent (risk factors) and dependent (ectopic pregnancy) variables without making any assumptions about the shape of that relationship. It still produced a smooth and easy-to-interpret curve like linear regression, but also avoided overfitting. The model's framework in predicting the risk of ectopic pregnancy utilized baseline β-hCG and the β-hCG ratio in combination, which gave the best AUC of 0.889. Applicant then included known risk factors and presenting symptoms for EP to strengthen the model for the outcome of EP. Applicant did not include gestational age because a majority of Applicant's patients did not know or were unsure of the date of their last menstrual periods. Applicant then calculated performance measures for a range of risk cut-offs for ectopic pregnancy. Based on Applicant's findings, a cut-off of 15% provided the best sensitivity and specificity (82.5%, 82.5%, respectively).

Adopting a risk threshold of 15%, clinicians would have an objective way to segregate their patients into a low-risk group if the score was <15% or a high-risk group if the score was >15%. Providers could adjust the intensity of monitoring based on this stratification. Although all patients would still be followed, the low-risk group, for example, could have less close observation than the high-risk group, thereby increasing efficiency and reducing cost as the low-risk patients would likely have less frequent visits and fewer lab draws. Furthermore, the Beta Book structure is used nationally at OB/GYN residency programs to manage PULs. Applicant's risk assessment tool has the potential to greatly reduce the time and resources used by OB/GYN residencies to manage these patients under the Beta Book protocol by developing new risk-stratified algorithms rather than using a single uniform approach to follow all patients with PULs.

Applicant's GAM model is a significant improvement over current diagnostic criteria for the detection of EP. It is novel in its approach as it allows for a risk calculation based on a comprehensive assessment of patient's clinical circumstances, which includes incorporation of historical factors and presenting symptoms as well as laboratory data. It can provide clinicians with a simple, objective and nuanced method for risk-stratifying their patients so that resources can be more thoughtfully allocated between groups based on risk. In one embodiment, the model can be implemented as a virtual application that clinicians can use to individualize patient care.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of estimating a probability of ectopic pregnancy in a subject, the computer-implemented method comprising:
receiving a parameter set comprising:
a first β-human chorionic gonadotropin (β-hCG) value generated using a first sample from a subject; and
a second β-human chorionic gonadotropin (β-hCG) value generated using a second sample from a subject, wherein the second sample is obtained between about 36 hours and about 72 hours after the first sample;
calculating a ratio the second (β-hCG value to the first β-hCG value to produce an augmented parameter set comprising:
the first β-hCG value; and
the ratio of the second (β-hCG value to the first β-hCG value; and
solving a generalized additive model for a probability of ectopic pregnancy in the subject based the augmented parameter set, wherein the generalized additive model was previously backfit with a data set of subject data including the augmented parameter set for each subject in the data set;
thereby estimating the probability of ectopic pregnancy in the subject.

2. The computer-implemented method of claim 1, wherein the generalized additive model is a penalized spline generalized additive model.

3. The computer-implemented method of claim 1, wherein the generalized additive model is a penalized log-likelihood generalized additive model.

4. The computer-implemented method of claim 1, wherein the parameter set and the augmented parameter set further comprise:
one or more risk factors.

5. The computer-implemented method of claim 4, wherein the one or more risk factors include one or more selected from the group consisting of: history of prior ectopic pregnancy, history of pelvic inflammatory disease, history of tubal ligation, presence of intrauterine device, history of diethylstilbestrol (DES) exposure, history of infertility, history of pelvic surgery, and history of sexually transmitted infections.

6. The computer-implemented method of claim 1, wherein the parameter set and the augmented parameter set further comprise:
one or more demographics.

7. The computer-implemented method of claim 6, wherein the one or more demographics include one or more selected from the group consisting of: age and parity.

8. The computer-implemented method of claim 1, wherein the parameter set and the augmented parameter set further comprise:
total time followed by medical professionals.

9. The computer-implemented method of claim 1, wherein the parameter set and the augmented parameter set further comprise:
total number of (β-hCG values measures.

10. The computer-implemented method of claim 1, wherein the first sample and the second sample are both selected from the group consisting of: blood, blood serum, blood plasma, and urine.

11. The computer-implemented method of claim 1, wherein the first β-hCG value to the second β-hCG value are determined using a sandwich assay.

12. The computer-implemented method of claim 1, wherein:
the first sample and the second sample are both urine; and
the first β-hCG value and the second β-hCG value are determined using an assay selected from the group consisting of: a chromatographic immunoassay and a lateral flow assay.

13. The computer-implemented method of claim 1, wherein:
the first sample and the second sample are both blood serum; and
the first β-hCG value and the second β-hCG value are determined using an assay selected from the group consisting of: a chemiluminescent immunoassay or fluorimetric immunoassay.

14. The computer-implemented method of any of claim 1, wherein the subject is mammal.

15. The computer-implemented method of claim 14, wherein the subject is a human female.

16. A non-transitory computer readable medium containing program instructions executable by a processor, the computer readable medium comprising program instructions to implement the computer-implemented method of claim 1.

17. A method of diagnosing and treating a subject presenting with a pregnancy of unknown location (PUL), the method comprising:
obtaining a parameter set comprising:
a first β-human chorionic gonadotropin (β-hCG) value generated using a first sample from a subject; and
a second β-human chorionic gonadotropin (β-hCG) value generated using a second sample from a subject, wherein the second sample is obtained between about 36 hours and about 72 hours after the first sample;
providing the parameter set as an input to either the computer-implemented method of claim 1 or a computer executing the program instructions of the non-transitory computer readable medium of claim 14;
receiving a probability of ectopic pregnancy in the subject; and
treating the subject based on the probability.

18. The method of claim 17, wherein the subject is mammal.

19. The method of claim 18, wherein the subject is a human female.
